(19) United States
(12) Patent Application Publication
(10) Pub. No.: US 2020/0009393 A1
(43) Pub. Date: Jan. 9, 2020

(54) Antenna Wire Termination Assemblies for Use in Implantable Medical Devices

(71) Applicant: Advanced Bionics AG, Staefa (CH)

(72) Inventor: Jeryle L. Walter, Valencia, CA (US)

(73) Assignee: Advanced Bionics AG

(21) Appl. No.: 16/026,179

(22) Filed: Jul. 3, 2018

(51) Int. Cl. A61N 1/372; A61N 1/36; A61N 1/375

(52) U.S. Cl. CPC A61N 1/37229; A61N 1/36038; A61N 1/362; A61N 1/36046; A61N 1/3754

(57) ABSTRACT

An exemplary antenna wire termination assembly includes a helically shaped conductive retention coil and a conductive tab member. The retention coil has a first end and a second end that define a length of the retention coil, a plurality of turns between the first and second ends, and a flat side along the length of the retention coil. The flat side is formed by a plurality of aligned straight segments included in the turns. The conductive tab member has a proximal portion conductively affixed to the flat side of the retention coil and a distal portion extending away from the second end of the retention coil in a direction that is parallel with the length of the retention coil.

### BACKGROUND INFORMATION

Implantable medical devices (e.g., cochlear implants, retinal implants, cardiac pacemakers, implantable defibrillators, recording devices, neurostimulators, etc.) often communicate with and/or receive power from an external device by way of a transcutaneous inductive link. To this end, an implantable medical device may include or be connected to an implantable antenna, which may form an inductive link with an external antenna that is connected to the external device.

During manufacturing of a conventional implantable medical device, conductive wires of an implantable antenna may be terminated in a manner that allows the wires to be conductively connected to a feedthrough electrical contact of a circuit included in the implantable medical device. To this end, distal ends of the conductive wires are conventionally placed within a tube, which is then crimped in order to hold the conductive wires in place. The tube is then conductively affixed to the feedthrough electrical contact. Unfortunately, this approach requires custom tooling and unique machined components. Moreover, it is difficult to manually hold the conductive wires in place while the tube is crimped. This results in manufacturing processes for implantable medical devices that can be relatively expensive, time consuming, and resource intensive.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary configuration in which an implantable medical device wirelessly communicates with an external device according to principles described herein.

FIG. 2 shows an exemplary cochlear implant according to principles described herein.

FIG. 3 shows an exemplary antenna according to principles described herein.

FIG. 4 illustrates an exemplary termination assembly according to principles described herein.

FIGS. 5A-5C show various views of a retention coil according to principles described herein.

FIG. 6 is a view of a bottom side of a tab member according to principles described herein.

FIG. 7 illustrates an exemplary method for connecting an antenna to a circuit included in an implantable medical device by way of termination assembly according to principles described herein.

FIG. 8 shows end portions of conductive wires positioned between turns of a termination assembly according to principles described herein.

FIGS. 9-10 show a retention coil positioned inside a can of an implantable medical device according to principles described herein.

FIG. 11 illustrates an exemplary implantable medical device assembly in which two termination assemblies are used to terminate and connect both ends of conductive wires included in an antenna to circuitry included in the implantable medical device assembly according to principles described herein.

### DETAILED DESCRIPTION

Antenna wire termination assemblies for use in implantable medical devices are described herein. For example, an exemplary antenna wire termination assembly (“termination assembly”) includes a helically shaped conductive retention coil and a conductive tab member. The retention coil has a first end and a second end that define a length of the retention coil, a plurality of turns between the first and second ends, and a flat side along the length of the retention coil. The flat side is formed by a plurality of aligned straight segments included in the turns. The conductive tab member has a proximal portion conductively affixed to the flat side of the retention coil and a distal portion extending away from the second end of the retention coil in a direction that is parallel with the length of the retention coil.

The termination assembly is configured to terminate a plurality of conductive wires of an antenna and conductively connect the wires to a feedthrough electrical contact of a circuit included in an implantable medical device. For example, end portions of the conductive wires may be positioned between the turns of the retention coil. The turns are configured to retain the end portions of the conductive wires. For example, a natural compressive force of the retention coil may be configured to retain the end portions of the conductive wires within the turns of the retention coil. Additionally or alternatively, the retention coil may be compressed by an external force (e.g., manually or by a machine) to retain the end portions of the conductive wires within the turns of the retention coil.

While the conductive wires are retained within the turns of the retention coil, the conductive wires are conductively affixed (e.g., welded) to the retention coil. This conductively couples the conductive wires to one another, to the retention coil, and to the conductive tab member. The distal portion of the tab member is conductively affixed (e.g., welded) to the feedthrough electrical contact of the circuit in the implantable medical device. In this manner, the conductive wires are conductively connected to the circuit. In instances where both ends of the conductive wires have to be terminated (e.g., for loop antennas, etc.), two termination assemblies as described herein may be used.

Various benefits are associated with the termination assemblies described herein. For example, the termination assemblies described herein obviate the need to crimp or weld a tube onto the ends of the conductive wires of an antenna, which obviates the need for custom tooling and/or machined components during manufacturing. Moreover, the termination assemblies described herein are self fixturing in that they hold the conductive wires of the antenna in place in between the turns of the termination assemblies even before the conductive wires are welded or otherwise affixed to the termination assemblies. This allows a user to more easily handle and assemble an implantable medical device during manufacturing. The termination assemblies described herein also provide strain relief and support for the conductive wires during testing that occurs during manufacturing and during normal use of the implantable medical device by a user. These and other benefits of the termination assemblies described herein will be made apparent in the description that follows.

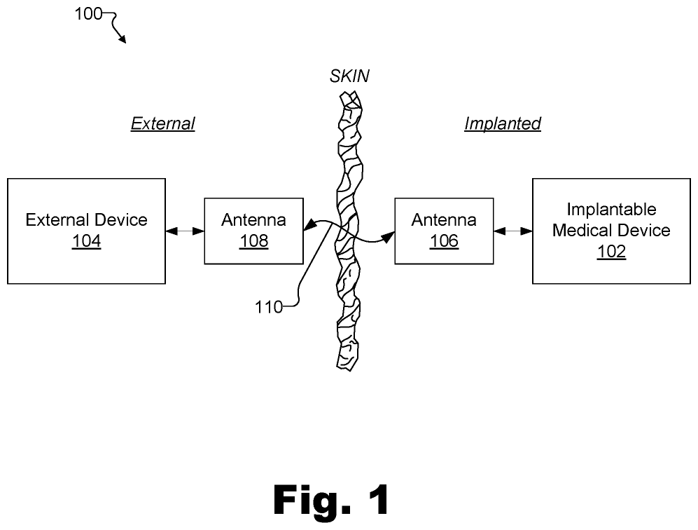

FIG. 1 illustrates an exemplary configuration 100 in which the termination assemblies described herein may be employed. As shown, configuration 100 includes an implantable medical device 102 configured to wirelessly (e.g., transcutaneously) communicate with an external device 104. To facilitate this communication, implantable medical device 102 is connected to an implanted antenna 106, and external device 104 is connected to an external antenna 108. Implanted antenna 106 may be separate from implantable medical device 102, as shown in FIG. 1. Alternatively implanted antenna 106 may be housed within the same housing (e.g., a hermetic can) as implantable medical device 102. Likewise, external antenna 108 may be separate from external device 104, as shown in FIG. 1. Alternatively, external antenna 108 may be housed within the same housing as external device 104.

Antennas 106 and 108 may form an inductive link 110 when in close proximity one to another. Signals (e.g., power and/or data) may be transmitted by external device 104 to implantable medical device 102 by way of inductive link 110. Signals (e.g., back telemetry signals) may additionally or alternatively be transmitted by implantable medical device 102 to external device 104 by way of inductive link 110.

Implantable medical device 102 and external device 104 may be implemented by any suitable combination of devices. For example, implantable medical device 102 may be implemented by a cochlear implant included in a cochlear implant system and external device 104 may be implemented by a sound processor included in the cochlear implant system. Implantable medical device 102 may alternatively be implemented by a retinal implant, a cardiac pacemaker, an implantable defibrillator, a recording device, a neurostimulator, and/or any other implantable medical device. External device 104 may alternatively be implemented by a remote control device, a mobile device (e.g., a mobile phone), a desktop computer, and/or any other computing or processing device configured to communicate with (e.g., control) implantable medical device 102.

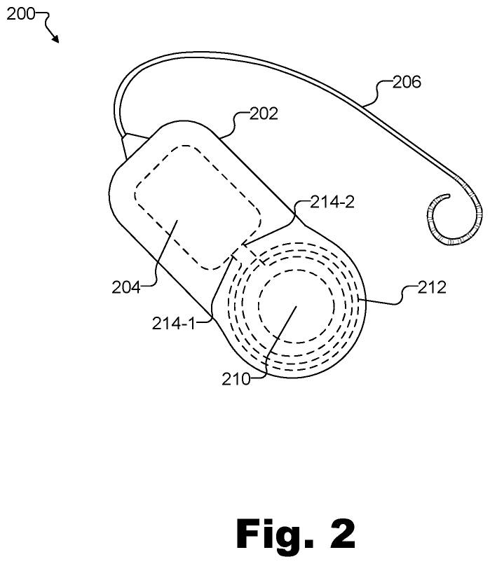

FIG. 2 shows an exemplary cochlear implant 200 that may implement implantable medical device 102. As shown, cochlear implant assembly 200 includes a flexible housing 202 formed from a silicon elastomer or other suitable material, a stimulation circuit 204 (which may be housed within a can or other suitable housing), a cochlear lead 206 with an electrode array 208, and a positioning element 210 (e.g., a magnet or other ferromagnetic material). As shown, an antenna 212 (which may implement antenna 106) is included in flexible housing 202 and connected to stimulation circuit 202. As shown, ends 214-1 and 214-2 of antenna 212 are coupled to stimulation circuit 204. As described herein, this coupling may be achieved by using the termination assemblies described herein. Stimulation circuit 204 may process signals (e.g., power and/or data signals) received by antenna 212.

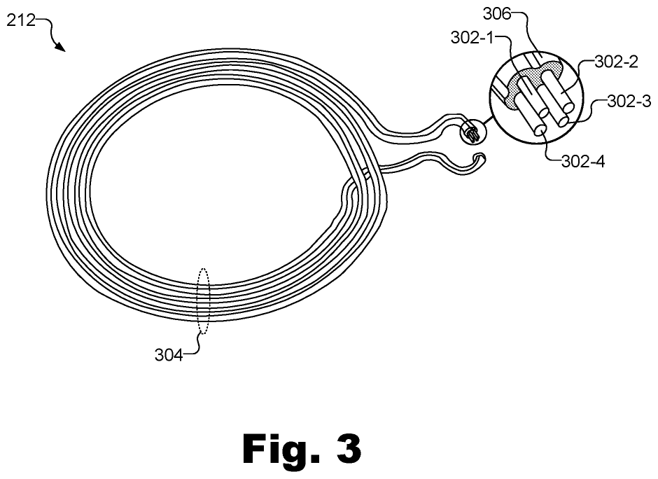

FIG. 3 shows antenna 212 in more detail. As shown, antenna 212 may include a plurality of conductive wires 302 (i.e., wire 302-1 through 302-4). In the example of FIG. 3, wires 302 form a plurality of loops 304. Hence, antenna 212 may be referred to as a loop antenna. Other types of antennas may alternatively be used. Exemplary antennas that may be included in or attached to implantable medical devices are described in U.S. Patent Publication No. 2018/0071542, the contents of which are incorporated herein by reference in their entirety.

Although four wires 302 are shown in FIG. 3, any number of wires may be included in antenna 212. Wires 302 may be made out of any suitable conductive material. As shown, wires 302 are located within a carrier 306 that maintains the position of wires 302 relative to one another. End portions of wires 302 are exposed to facilitate conductive connection of wires 302 to circuit 204 (e.g., to a feedthrough electrical contact of circuit 204).

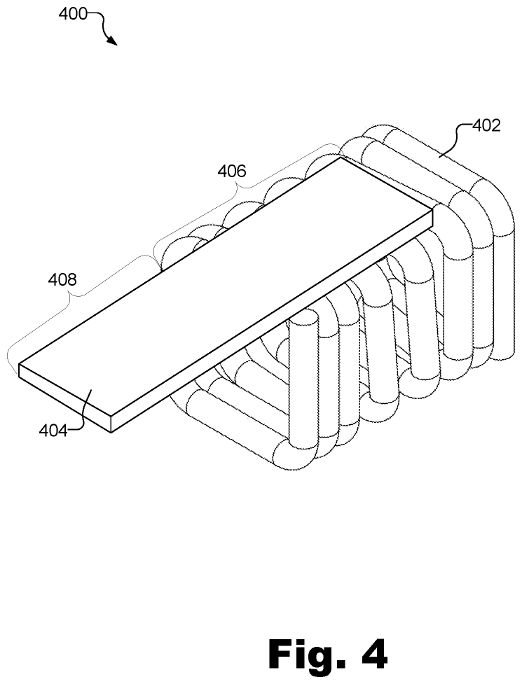

FIG. 4 illustrates an exemplary termination assembly 400 that may be used to terminate a plurality of conductive wires (e.g., wires 302) of an antenna (e.g., antenna 212) and conductively connect the wires to a feedthrough electrical contact of a circuit included in an implantable medical device. As shown, termination assembly 400 includes a helically shaped conductive retention coil 402 and a conductive tab member 404. A proximal portion 406 of tab member 404 is conductively affixed to a flat side (e.g., a top flat side, as described below) of retention coil. A distal portion 408 of tab member 404 extends away from an end of retention coil 402 in a direction that is parallel with a length of retention coil 402.

Retention coil 402 and tab member 404 may each be made out of any suitable conductive material (e.g., a conductive metal). As will be described below, while FIG. 4 shows that tab member 404 is affixed to a top side of retention coil 402, it will be recognized that tab member 404 may not be initially affixed to retention coil 402 when wires of an antenna are placed between the turns of retention coil 402.

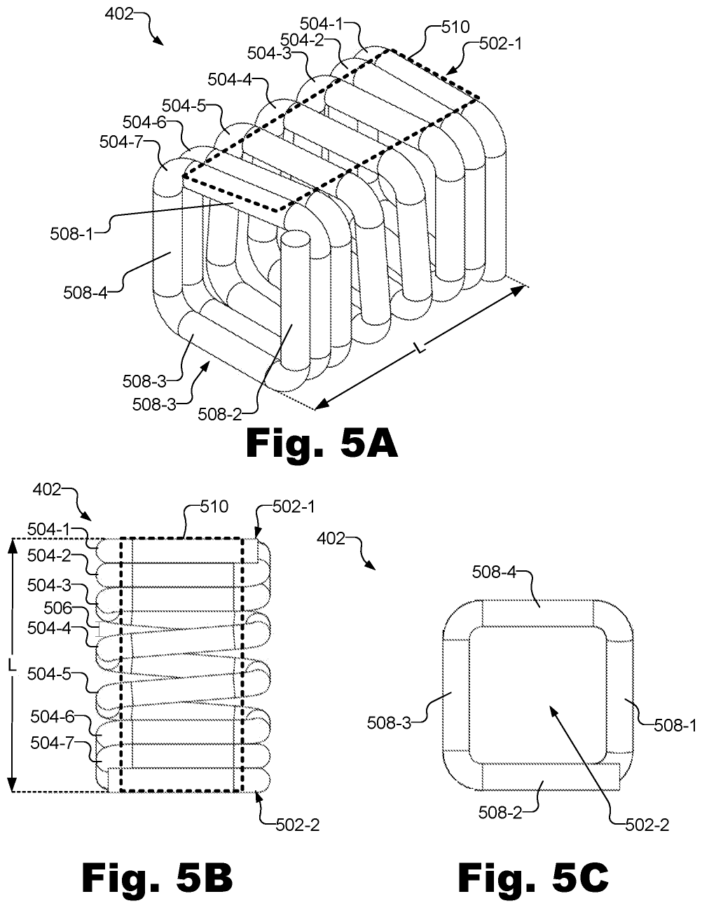

FIGS. 5A-5C show various views of retention coil 402. In particular, FIG. 5A shows a perspective side view of retention coil 402, FIG. 5B shows a top view of retention coil 402, and FIG. 5C shows an end view of retention coil 402. As shown in one or more of these views, retention coil 402 includes a first end 502-1 and a second end 502-2 (collectively “ends 502”) that define a length L of retention coil 402.

Retention coil 402 further includes a plurality of turns 504 (e.g., turns 504-1 through 504-7) between ends 502. Turns 504 may be formed by round wire, as shown in FIGS. 5A-5C. Alternatively, turns 504 may be formed by square wire and/or wire of any other suitable shape.

As will be described below, turns 504 are configured to retain end portions of a plurality of conductive wires of an antenna that are positioned between 504. To this end, as labeled in FIG. 5B, retention coil 402 may be made to initially (i.e., before retention coil 402 is compressed) have spacing (e.g., spacing 506) between at least some adjacent turns (e.g., between turns 504-3 and 504-4). Alternatively, the spacing may be created by pulling adjacent turns 504 apart (e.g., either manually or with a tool or machine). In some examples, retention coil 402 exhibits one or more spring-like attributes. For example, retention coil 402 may stretched or compressed and then return to its natural resting state.

As shown, each turn 504 includes a plurality of straight segments. For example, turn 504-7 includes straight segments 508-1 through 508-4. In the particular example of FIGS. 5A-5C, the straight segments of each turn 504 forms a helical square shape. Hence, retention coil 402 may be referred to as a square (or rectangular) retention coil. Other retention coils 402 that have turns that include at least one straight segment may alternatively be used. For example, alternative retention coils may be triangular (i.e., each turn may have three straight segments), semi-circular (i.e., each turn may include a semicircle and a single straight segment), etc.

As shown, because retention coil 402 is square shaped, retention coil 402 has four flat sides each formed by a plurality of aligned straight segments included in turns 504. For example, a dashed box shown in FIGS. 5A and 5B outlines a top flat side 510 of retention coil. Top flat side 510 is formed by straight segment 508-1 of turn 504-7 and a plurality of other straight segments included in the other turns 504-1 through 504-6. As will be described below, top flat side 510 may advantageously provide a relatively large surface to which tab member 404 may be affixed. A bottom flat side (not labeled in FIGS. 5A-5C), but opposite top flat side 510, may be configured to be seated within a cavity of a can of an implantable medical device.

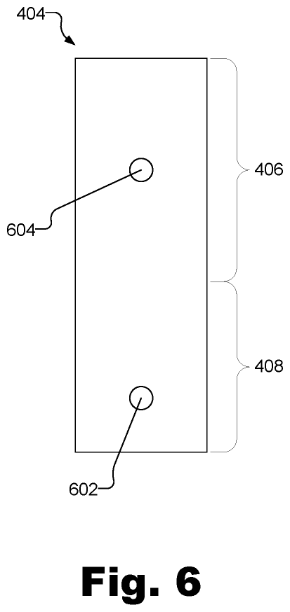

FIG. 6 is a view of a bottom side of tab member 404. As mentioned, proximal portion 406 of tab member 402 may be conductively affixed to top flat side 510 of retention coil 402, while distal portion 408 of tab member 402 may extend past end 508-2 of retention coil 402.

As shown, distal portion 408 includes a conductive receptacle 602. Conductive receptacle 602 is positioned to align with and couple to a feedthrough electrical contact (e.g. a feedthrough pin) of a circuit of the implantable medical device when termination assembly 400 is placed within a can of the implantable medical device. Tab member 404 may be configured to conductively connect to the circuit in any other suitable manner.

As also shown, proximal portion 406 also includes a conductive receptacle 604. Conductive receptacle 604 is configured to facilitate affixation (e.g., welding) of tab member 404 to top flat side 510 of retention coil 402. In some alternative embodiments, conductive receptacle 604 is not included on tab member 404.

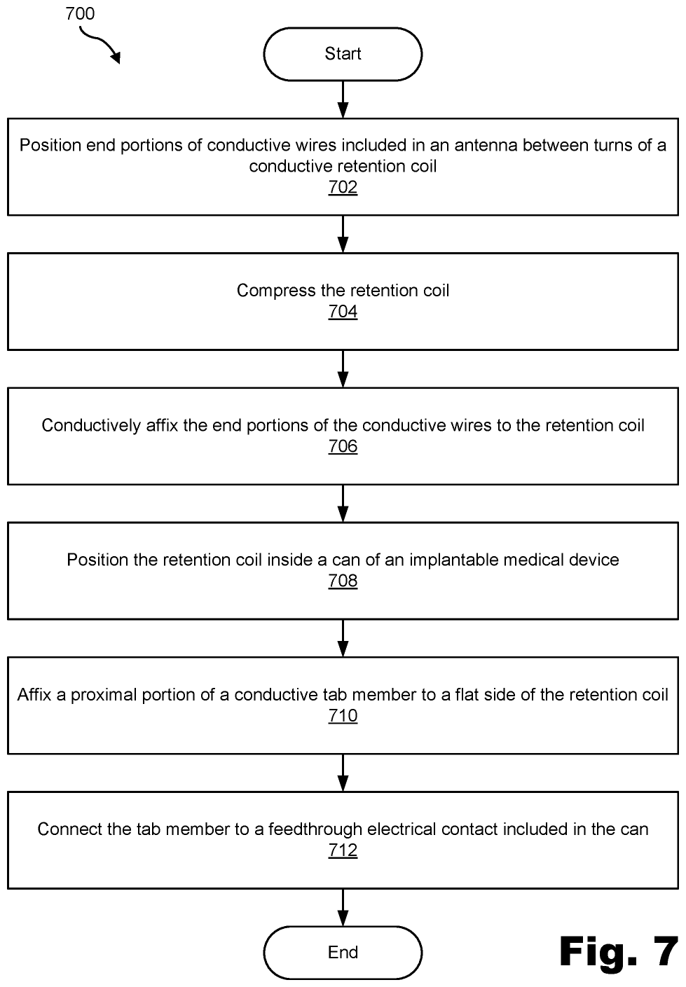

FIG. 7 illustrates an exemplary method 700 for connecting an antenna to a circuit included in an implantable medical device by way of termination assembly 402. While FIG. 7 illustrates exemplary steps according to one embodiment, other embodiments may omit, add to, reorder, and/or modify any of the operations shown in FIG. 7.

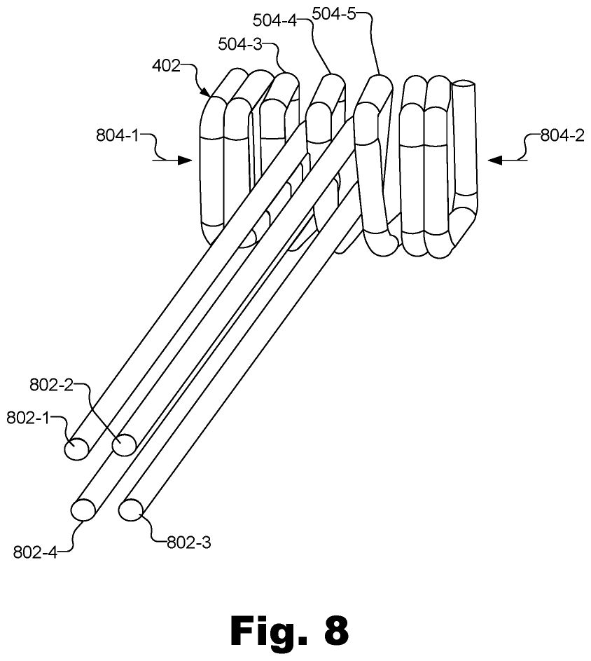

In step 702, end portions of a plurality of conductive wires included in an antenna are positioned between turns 504 of termination assembly 402. For example, FIG. 8 shows that end portions of conductive wires 802 (i.e., wires 802-1 through 802-4) are positioned between turns 504-3 through 504-5 of termination assembly 402. In particular, conductive wires 802-1 and 802-4 are positioned between turns 504-3 and 504-4, and conductive wires 802-4 and 802-5 are positioned between turns 504-4 and 504-5. Conductive wires 802 may be positioned between any of the other turns 504 of termination assembly 402 as desired.

End portions of wires 802 may be positioned between turns 504 of termination assembly 402 in any suitable manner. For example, a person may manually position wires 802 between turns 504 of termination assembly 402. In some examples, the person may manually separate turns 504 (e.g., by pulling turns 504 apart) in order to fit wires 802 therebetween. Alternatively, the spacing between turns 504 may be such that wires 802 may be readily positioned between turns 504 without requiring manual separation of turns 502. In some alternative embodiments, a tool or machine is used to position wires 802 between turns 504.

In step 704, retention coil 402 is compressed. For example, compressive force may be applied in the direction of arrows 804-1 and 804-2 to compress retention coil 402. This compression may retain wires 802 between turns 504. The compression of retention coil 402 may be performed manually (e.g., by a person squeezing the two ends of retention coil 402 towards each other) and/or with a tool or machine.

In some alternative embodiments, step 704 is omitted (i.e., not performed). For example, if retention coil 402 sufficiently retains wires 802 between turns 504, it may not be necessary to compress retention coil 402.

In step 706, the end portions of wires 802 are conductively affixed to retention coil 402. For example, once wires 802 have been positioned between turns 504, wires 802 may be welded (e.g., resistance welded or laser welded), soldered, bonded with conductive epoxy, or otherwise conductively coupled to retention coil 402 (i.e., between turns 504). In this manner, wires 802 may be even more securely connected to retention coil 402.

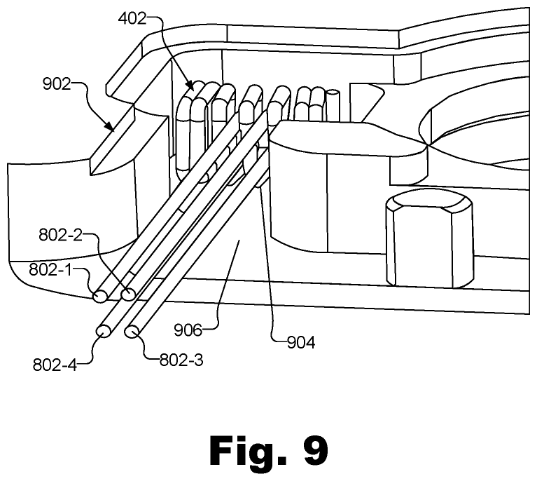

In step 708, retention coil 402 is positioned inside a can of an implantable medical device. To illustrate, FIG. 9 shows retention coil 402 positioned inside a can 902 of an implantable medical device. Can 902 may be implemented by any suitable housing configured to house various components (e.g., a circuit) of the implantable medical device. As shown, a bottom flat side 904 of retention coil 402 is seated on a floor 906 of can 902.

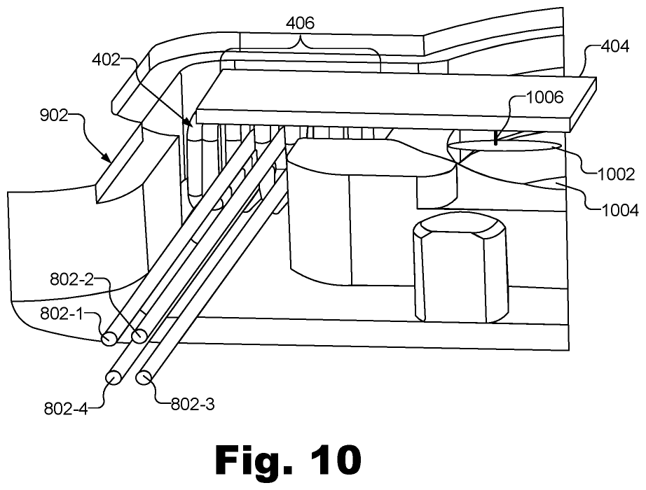

In step 710, proximal portion 406 of tab member 404 is affixed to the top flat side of retention coil 402. This is shown in FIG. 10. Proximal portion 406 may be welded (e.g., resistance welded or laser welded), soldered, bonded with conductive epoxy, or otherwise conductively coupled to the top flat side of retention coil 402. In some alternative examples, step 710 is performed prior to step 708.

In step 712, tab member 404 is connected to a feedthrough electrical contact included in can 902. For example, with reference to FIG. 10, a circuit 1002 may be positioned within cavity 1004 of can 902. Circuit 1002 may include a feedthrough electrical contact 1006 (e.g., a feedthrough pin) that points upward such that it may be coupled with conductive receptacle 602 located on the bottom side of tab member 404. Once feedthrough electrical contact 1006 is inserted into conductive receptacle 602, feedthrough electrical contact 1006 may be affixed (e.g., welded, soldered, etc.) to conductive receptacle 602.

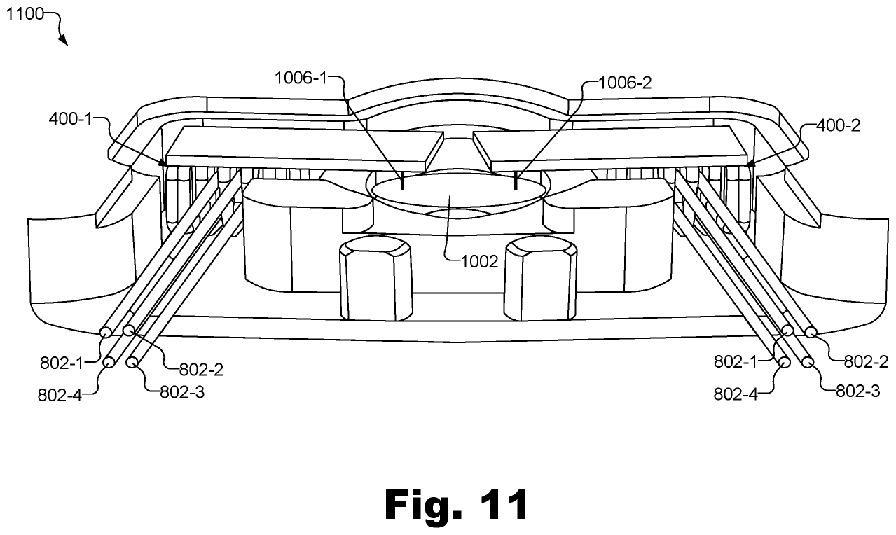

FIG. 11 illustrates an exemplary implantable medical device assembly 1100 in which two termination assemblies are used to terminate and connect both ends of conductive wires included in an antenna to circuitry included in the implantable medical device assembly. As shown, a first termination assembly 400-1 retains a first end portion of conductive wires 802-1 through 802-4 and couples conductive wires 802-1 through 802-4 to circuit 1002 by way of feedthrough electrical contact 1006-1. Likewise, a second termination assembly 400-2 retains a second end portion of conductive wires 802-1 through 802-4 and couples conductive wires 802-1 through 802-4 to circuit 1002 by way of feedthrough electrical contact 1006-2. Termination assemblies 400-1 and 400-2 may each be similar to termination assembly 400 as described herein.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A termination assembly comprising:
a helically shaped conductive retention coil having
a first end and a second end that define a length of the retention coil,
a plurality of turns between the first and second ends, the turns configured to retain end portions of a plurality of conductive wires of an antenna that are positioned between the turns, and
a flat side along the length of the retention coil, the flat side formed by a plurality of aligned straight segments included in the turns; and
a conductive tab member having
a proximal portion conductively affixed to the flat side of the retention coil, and
a distal portion extending away from the second end in a direction that is parallel with the length of the retention coil.

2. The termination assembly of claim 1, wherein the distal portion of the conductive tab member is configured to conductively connect to a circuit configured to process signals received by the antenna.

3. The termination assembly of claim 2, wherein the distal portion of the conductive tab member comprises a conductive receptacle configured to be conductively coupled to a feedthrough electrical contact of the circuit.

4. The termination assembly of claim 2, wherein the circuit is included in an implantable medical device.

5. The termination assembly of claim 3, wherein the implantable medical device is a cochlear implant.

6. The termination assembly of claim 1, wherein the retention coil conductively couples the conductive wires one to another.

7. The termination assembly of claim 1, wherein the retention coil is compressed to retain the end portions of the conductive wires.

8. The termination assembly of claim 1, wherein the end portions of the conductive wires are conductively affixed to the retention coil.

9. The termination assembly of claim 1, wherein:
the plurality of conductive wires comprises a first conductive wire, a second conductive wire, a third conductive wire, and a fourth conductive wire;
the end portions of the first and second conductive wires are positioned between a first turn included in the plurality of turns and a second turn included in the plurality of turns; and
the end portions of the third and fourth conductive wires are positioned between the second turn and a third turn included in the plurality of turns.

10. The termination assembly of claim 1, wherein:
the end portions of the conductive wires are welded between the turns of the retention coil; and
the proximal portion of the tab member is welded to the flat side of the retention coil.

11. The termination assembly of claim 1, wherein the retention coil further comprises an additional flat side along the length of the retention coil, the additional flat side configured to be seated within a cavity of a can of an implantable medical device.

12. The termination assembly of claim 1, wherein the retention coil is rectangular.

13. An implantable medical device assembly comprising:
a can;
a circuit housed within the can and that is configured to process signals received from an external device;
a helically shaped conductive retention coil housed within the can and that has
a first end and a second end that define a length of the retention coil,
a plurality of turns between the first and second ends, and
a flat side along the length of the retention coil, the flat side formed by a plurality of aligned straight segments included in the turns;
an antenna having a plurality of conductive wires, the conductive wires each having an end portion positioned between and retained by the turns of the retention coil; and
a conductive tab member having
a proximal portion conductively affixed to the flat side of the retention coil, and
a distal portion extending away from the second end in a direction that is parallel with the length of the retention coil, the distal portion conductively connected to the circuit.

14. The implantable medical device assembly of claim 13, further comprising:
an additional helically shaped conductive retention coil housed within the can and that has
a first end and a second end that define a length of the additional retention coil,
a plurality of turns between the first and second ends of the additional retention coil, and
a flat side along the length of the additional retention coil, the flat side of the additional retention coil formed by a plurality of aligned straight segments included in the turns of the additional retention coil; and
an additional conductive tab member having
a proximal portion conductively affixed to the flat side of the additional retention coil, and
a distal portion extending away from the second end of the additional retention coil in a direction that is parallel with the length of the additional retention coil;
wherein the conductive wires each have an additional end portion positioned between and retained by the turns of the additional retention coil.

15. The implantable medical device assembly of claim 13, wherein the distal portion of the additional conductive tab member is configured to conductively connect to the circuit.

16. The implantable medical device assembly of claim 13, wherein the retention coil is compressed to retain the end portions of the conductive wires.

17. A method comprising:
positioning end portions of a plurality of conductive wires included in an antenna between turns of a helically shaped conductive retention coil, the retention coil having
a first end and a second end that define a length of the retention coil, and
a flat side along the length of the retention coil, the flat side formed by a plurality of aligned straight segments included in the turns;
conductively affixing the end portions of the conductive wires to the retention coil; and
affixing a proximal portion of a conductive tab member to the flat side of the retention coil.

18. The method of claim 17, further comprising positioning the retention coil inside a can of an implantable medical device.

19. The method of claim 18, further comprising connecting the conductive tab member to a feedthrough electrical contact included in the can.

20. The method of claim 17, further comprising compressing the retention coil prior to affixing the end portions of the conductive wires to the retention coil.
